(19) United States
(12) Patent Application Publication
(10) Pub. No.: US 2010/0015710 A1
(43) Pub. Date: Jan. 21, 2010

(54) Methods and Compositions for Isolating, Maintaining and Serially Expanding Human Mesenchymal Stem Cells

(76) Inventors: Sunghoon Jung, Calgary (CA); Arindom Sen, Calgary (CA); Leo A. Behie, Calgary (CA)

Correspondence Address: FULBRIGHT & JAWORSKI L.L.P., 600 CONGRESS AVE., SUITE 2400, AUSTIN, TX, 78701, US

(21) Appl. No.: 12/430,674

(22) Filed: Apr. 27, 2009

Related U.S. Application Data
(60) Provisional application No. 61/048,017, filed on Apr. 25, 2008.

(51) Int. Cl. C12N 5/00; C12M 1/00

(52) U.S. Cl. 435/377; 435/405; 435/304.1; 435/305.1; 435/384

(57) ABSTRACT

Compositions and methods for isolating and expanding human mesenchymal stem/progenitor cells through multiple passages in defined serum-free environments are provided. The culture media compositions includes a basal medium supplemented with a nutrient mixture such as Ham's F12 nutrient mixture, glutamine, buffer solutions such as sodium bicarbonate and hepes, serum albumin, a lipid mixture, insulin, transferrin, putrescine, progesterone, fetuin, hydrocortisone, ascorbic acid or its analogues such as ascorbic acid-2-phosphate, fibroblast growth factor and transforming growth factor β, and are free of serum or other undefined serum substitutes such as platelet lysate. Methods employing these compositions and protein-coated surfaces for the isolation of mesenchymal stem/progenitor cells from human bone marrow and other tissues such as adipose tissue are also provided. Finally, methods are also provided for serially expanding these cells through multiple passages without losing mesenchymal stem cell-specific proliferative, phenotypical and differentiation characteristics.

The present application claims benefit of priority to U.S. Provisional Application Ser. No. 61/048,017, filed Apr. 25, 2008, the entire contents of which are hereby incorporated by reference.

### BACKGROUND OF THE INVENTION

1. Field of the Invention

The present invention relates generally to the fields of cell biology and stem cell bioengineering. More particularly, it concerns methods and compositions for culturing mesenchymal stem/progenitor cells. The term culturing includes isolating, maintaining and serially expanding mesenchymal stem/progenitor cells.

2. Description of the Related Art

Mesenchymal stem cells (MSCs) refer to cells that have the potential to self-renew and are capable of differentiating into multiple mesenchymal lineages (Pittenger et al., 1999). Also, recent findings show that these cells may have the capacity to give rise to other germ layer cell types such as neuronal cells (Tropel et al., 2007). The major source of human MSCs (hMSCs) is bone marrow from which they are safely and readily isolated. These cells can also be isolated from other tissues such as adipose tissue. Due to their scarcity in adult tissues, hMSCs can normally only be isolated in small numbers; however, they have an extensive capacity for proliferation and can be readily expanded in culture to generate clinically-relevant numbers of cells through multiple passages. In addition to their multi-lineage differentiation potential, studies also show that hMSCs secrete bioactive factors that are immunoregulatory and/or support tissue repair and regeneration (Caplan, 2007). These properties, together with their extensive proliferative capacity in culture, have made hMSCs a promising tool for various therapeutic applications.

Conventional media used for isolating and expanding hMSCs typically consist of a defined basal medium (e.g., DMEM or α-MEM) supplemented with various concentrations (10-20% v/v) of fetal bovine serum (FBS) due to its high content of stimulatory growth factors. Although these media are generally reported to support the proliferation of hMSCs for multiple passages, and clinical studies involving hMSCs expanded in the presence of FBS have already started, concerns have been raised because of the potential risks associated with FBS [Dimarakis and Levicar, 2006; Mannello and Tonti, 2007]. FBS may contain harmful contaminants such as prion, viral, or zoonotic agents, and can elicit immune reactions. Moreover, the poorly defined nature of FBS, and its high degree of batch-to-batch variation can cause inconsistencies in the growth-supporting properties of a medium, and thus makes standardization of a cell production process difficult. Furthermore, undefined components in FBS can interfere with the effects of growth factors or hormones when studying their interactions with cells, thereby making the interpretation of experiments carried out in FBS-containing media difficult. Thus, the use of FBS represents a major obstacle for the clinical implementation of hMSC-related therapies.

Although human-sourced supplements such as human serum, plasma, or platelet lysate have been investigated to replace FBS [Stute//Zander et al., 2004; Doucet//Lataillade et al., 2005; Müller//Dominici et al., 2006; Capelli//Introna et al., 2007; Le Blanc//Ringdén et al., 2007; Lange//Zander et al., 2007], they are also ill defined. A more attractive alternative would be a defined serum-free medium. Efforts have been made to develop defined serum-free media for the expansion of MSCs from human (U.S. Pat. No. 5,908,782; U.S. Patent 2005/0265980 A1; U.S. Pat. No. 7,109,032 B2) and rat (Lennon et al., 1995) bone marrow, and other human tissues such as cord blood (Liu et al., 2007) and adipose tissue (Parker et al., 2007). However, their formulations were only able to support a slow rate of cell growth, and/or only for a limited number of passages. Moreover, these studies all used cells which had previously been exposed to serum during the initial isolation/expansion phases, and none of the serum-free media reported supported the derivation of MSCs from primary tissues in the absence of serum. Therefore, it is desirable to develop defined serum-free media and methods for (i) isolating hMSCs and (ii) expanding these cells for an extended period of time through multiple passages while maintaining their multi-lineage differentiation potential. The creation of defined serum-free media and methods should provide a robust platform that will help to enable the clinical implementation of hMSC-based therapies.

### SUMMARY OF THE INVENTION

Thus, in accordance with the present invention, there is provided a culture medium comprising, dissolved or dispersed in base culture medium and water, the following components:
(a) nutrients;
(b) glutamine;
(c) sodium bicarbonate;
(d) hepes;
(e) serum albumin;
(f) lipids;
(g) insulin;
(h) transferrin;
(i) putrescine;
(j) progesterone;
(k) fetuin or α₂-macroglubulin;
(l) hydrocortisone;
(m) ascorbic acid;
(n) bFGF; and
(o) TGF-β1.
   The base culture medium may be MCDB media series, CMRL medium-1066, Roswell Park Memorial Institute (RPMI) medium, alpha Modified Eagle's Medium (α-MEM), Dulbecco's Modified Eagle's Medium (DMEM), or Iscove's Modified Dulbecco's Medium (IMDM). The nutrients may comprise Ham's F12 nutrient mixture. The medium may be filter sterilized.

In particular embodiments, sodium bicarbonate is present at about 0.1 g/L to about 4 g/L, Hepes is present at about 1 mM to about 10 mM, serum albumin is present at about 0.1 g/L to about 10 g/L, fetuin is present at about 0.1 g/L to about 10 g/L, α₂-macroglubulin is present at about 0.4 mg/L to about 40 mg/L, hydrocortisone is present at about 1 nM to about 1000 nM, ascorbic acid 2 is present at about 1 μM to about 1000 μM, and lipids are present at about 0.1 mL of lipid concentrate/L to about 10 mL of lipid concentrate/L. In more particular embodiments, sodium bicarbonate is present at about 1.725 g/L, Hepes is present at about 4.9 mM, serum albumin is present at about 4 g/L, fetuin is present at about 1 g/L, hydrocortisone is present at about 100 nM, ascorbic acid is present at about 198 μM, and lipids are present at about 1 mL of lipid concentrate/L.

In particular embodiments, bFGF is present at about 0.01 μg/L to about 100 μg/L, and TGF-β1 is present at about 0.01 μg/L to about 100 μg/L. In more particular embodiments, bFGF is present at about 0.1 μg/L to about 20 μg/L, and TGF-β1 is present at about 0.1 μg/L to about 20 μg/L. In even more particular embodiments, bFGF is present at about 2.0 μg/L, and TGF-β1 is present at about 1.0 μg/L.

In particular embodiments, transferrin is present at about 0.01 mg/L to about 100 mg/L, insulin is present at about 0.01 mg/L to about 100 mg/L, putrescine is present at about 0.01 mg/L to about 100 mg/L, and progesterone is present at about 0.001 μg/L to about 100 μg/L. In more particular embodiments, transferrin is present at about 10 mg/L to about 40 mg/L, insulin is present at about 10 mg/L to about 40 mg/L, putrescine is present at about 5 mg/L to about 20 mg/L, and progesterone is present at about 0.1 μg/L to about 20 μg/L. In even more particular embodiments, transferrin is present at about 25 mg/L, insulin is present at about 23 mg/L, putrescine is present at about 9 mg/L, and progesterone is present at about 5.66 μg/L.

In another embodiment, there is provided a cell culture container comprising at least one cell and the medium of claim 1. The cell may be a mesenchymal stem cell, also known as mesenchymal stromal cell, multipotent stromal cell, multipotent mesenchymal stromal cell, mesenchymal progenitor cell, or colony forming unit-fibroblast. The container may be a dish, flask, vessel, bottle or multi-well plate, and may be coated with a protein.

In still another embodiment, there is provided a method of culturing a mesenchymal stem cell comprising the steps of (a) providing a mesenchymal stem cell or mesenchymal stem cell-containing population in culture medium in a container; and (b) culturing said mesenchymal stem cell population in the culture medium as described above under conditions that produce a monolayer of cells adhered to a surface. The mesenchymal stem cell or mesenchymal stem cell-containing population may retain a mesenchymal stem or progenitor cell marker. The mesenchymal stem cell or mesenchymal stem cell-containing population may be obtained from bone marrow and other tissues such as adipose tissue. The mesenchymal stem cell or mesenchymal stem cell-containing population may be passaged 1, 2, 3, 4, 5, 6, 7, 8, 9, 10, 12, 14, 16, 18, 20, 22 times or more. The mesenchymal stem cell or mesenchymal stem cell-containing population may be maintained in culture for 2, 3, 4, 5, 6, 7, 8, 9, 10, 20, 25, 30, 35, 40, 45, 50, 60, 70, 80, 90 days or more. The method may further comprise inducing differentiation of said mesenchymal stem cell or mesenchymal stem cell-containing population. The mesenchymal stem cell or mesenchymal stem cell-containing population may differentiate into cells of the adipogenic lineage and/or osteogenic lineage and/or chondrogenic lineage, or into an adipocyte(s) and/or an osteoblast(s) and/or a chondroblast(s). The mesenchymal stem cell or mesenchymal stem cell-containing population may be maintained at about 75-99% viability, or at about 90-99% viability. The mesenchymal stem cell or mesenchymal stem cell-containing population may be cultured in a stationary phase. The cell-fold expansion may be 1-10²⁰ for the first 60 days or more. The method may further provide for the isolation of said mesenchymal stem cells prior to culturing.

The embodiments in the Examples section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value, and may also be interpreted at ±10% of a stated value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-B—CFU-F Assay of Primary Human Bone Marrow Mononuclear Cells (Defined Serum-Free Medium versus FBS-Supplemented Medium. Figures show (FIG. 1A) frequency of colony forming unit-fibroblast (CFU-F) in two separate donor human bone marrow mononuclear cells (BM MNCs; BM1 and BM2) determined by a limiting dilution assay using a standard FBS-supplemented medium (i.e., 10% FBS DMEM) from Lonza (formerly Cambrex Biosciences, Walkersville, Md.) versus the defined serum-free medium of the present invention (i.e., PPRF-msc6 medium), and (FIG. 1B) the colonies developed for 12 days at different seeding densities of BM MNCs.

FIGS. 2A-D—Colony Formation from BM MNC's in PPRF-msc6. Figures show photomicrographs (5×) presenting the formation of colonies (FIGS. 2A-B; days 5-10) and various individual colonies (FIGS. 2C-D; day 10) from the primary culture of human BM MNCs in 10% FBS DMEM (FIGS. 2A and 2C) versus PPRF-msc6 (FIGS. 2B and 2D).

FIG. 3—Isolation and Expansion of Human Bone Marrow-Derived Mesenchymal Stem Cells (BM-hMSC's) in PPRF-msc6 versus 10% FBS DMEM. Figure shows growth kinetics of human bone marrow-derived mesenchymal stem cells (BM-hMSCs) isolated from two separate donor BM MNCs (BM1 and BM2) and serially expanded in PPRF-msc6 versus 10% FBS DMEM.

FIG. 4—Morphology of BM-hMSC's Expanded in PPRF-msc6 versus 10% FBS DMEM. Figure shows phase contrast photomicrographs of BM-hMSCs (at passage levels 2, 4, and 6) isolated and serially expanded in PPRF-msc6 versus 10% FBS DMEM.

FIG. 5—Colony-Frequency of BM-hMSC's Isolated and Expanded in PPRF-msc6 versus 10% FBS DMEM. Figure shows CFU-F frequency of BM-hMSCs, which were previously isolated and expanded up to 4 passages in PPRF-msc6 versus 10% FBS DMEM, seeded at 100 cells/dish into 60 cm² dishes using the medium in which they had been expanded, and allowed to grow for 2-3 weeks.

FIG. 6—CFU-F Assay of BM-hMSC's Isolated and Expanded in PPRF-msc6 versus 10% FBS DMEM. Figure shows colonies derived from BM-hMSCs, which were previously isolated and expanded up to 4 passages in PPRF-msc6 versus 10% FBS DMEM, seeded at 100 cells/dish into 60 cm² dishes using the medium in which they had been expanded, and stained on days 14, 17 and 24.

FIGS. 7A-B—Individual Colonies Derived from BM-hMSC's Isolated and Expanded in PPRF-msc6 versus 10% FBS DMEM. Figure show photomicrographs representing individual colonies formed during CFU-F assay of BM-hMSCs expanded in PPRF-msc6 (FIG. 7A) versus 10% FBS DMEM (FIG. 7B).

FIG. 8—Flow Cytometry Analysis of BM-hMSC's at Passage 3 (P3). Figure shows flow cytometry analysis of phenotypes of BM-hMSCs isolated and expanded up to 3 passages in PPRF-msc6 versus 10% FBS DMEM. Expression of HLA-DR molecules by the activation with IFN-γ is also shown in the panel (solid with dark green).

FIGS. 9A-C—Adipogenic Potential of BM-hMSC's Isolated and Expanded in PPRF-msc6 versus 10% FBS DMEM. present adipogenic potential of BM-hMSCs isolated and expanded up to 4 passages in PPRF-msc6 versus 10% FBS DMEM, showing the intracellular lipids formed in the induced cultures and stained with AdipoRed assay reagent (FIG. 9A—bright field; FIG. 9B—fluorescent field) and the relative fluorescence (FIG. 9C).

FIGS. 10A-C—Osteogenic Potential of BM-hMSC's Isolated and Expanded in PPRF-msc6 versus 10% FBS DMEM. Figures present osteogenic potential of BM-hMSCs isolated and expanded up to 4 passages in PPRF-msc6 (versus 10% FBS DMEM), showing the induced (FIG. 10A) and non-induced (FIG. 10B) cultures stained with Alizarin Red S to detect calcium deposition and (FIG. 10C) the quantification of the deposition.

FIG. 11—Isolation and Expansion of Human Adipose Tissue-Derived MSC's (AT-hMSC's) in PPRF-msc6 versus 10% FBS DMEM. Figure shows growth kinetics of human adipose tissue-derived mesenchymal stem cells (AT-hMSCs) isolated from two separate donor tissues (AT1 and AT2) and expanded through multiple passages in PPRF-msc6 versus 10% FBS DMEM.

FIGS. 12A-B—Morphology of AT-hMSC's Isolated and Expanded in PPRF-msc6. Figures show phase contrast photomicrographs of AT-hMSCs grown in PPRF-msc6 medium in (FIG. 12A) primary culture (in comparison with cells grown in 10% FBS DMEM) and (FIG. 12B) passaged cultures.

FIG. 13—Flow Cytometry Analysis of AT-hMSCs at Passage 5 (P5) grown in PPRF-msc6. Figure shows flow cytometry analysis of phenotypes of AT-hMSCs isolated and expanded up to 5 passages in PPRF-msc6.

FIG. 14—Adipogenic and Osteogenic Potential of AT-hMSC's Isolated and Expanded in PPRF-msc6. Figure presents adipogenic and osteogenic potential of AT-hMSCs isolated and expanded up to 5 passages in PPRF-msc6.

FIG. 15—Comparison of the Impact of Recombinant Human Insulin (in PPRF-msc6h) versus Bovine Insulin (in PPRF-msc6) on the Isolation and Expansion of BM-hMSC's. Figure shows growth kinetics of BM-hMSCs isolated from BM MNCs and serially expanded in PPRF-msc6 (containing bovine insulin) versus PPRF-msc6h (containing recombinant human insulin) under two different substrate conditions, i.e., gelatin (bovine)-coated and fibronectin (human)-coated surface. A FBS-based control culture was also maintained with the use of gelatin-coated substrate.

FIG. 16—Comparison of the Impact of Recombinant Human Insulin (in PPRF-msc6h) versus Bovine Insulin (in PPRF-msc6) on the Isolation and Expansion of AT-hMSCs. Figure shows growth kinetics of AT-hMSCs isolated from adipose tissue and serially expanded in PPRF-msc6 (containing bovine insulin), PPRF-msc6h (containing recombinant human insulin) versus FBS-supplemented control medium on fibronectin (human)-coated surface.

### DESCRIPTION OF THE ILLUSTRATIVE EMBODIMENTS

In this study, the inventors report successful isolation and serial expansion (through multiple passages) of MSCs from human bone marrow and adipose tissue without losing mesenchymal stem cell-specific phenotypical, proliferative, and differentiation characteristics. The work was conducted at the Pharmaceutical Production Research Facility (PPRF) at the University of Calgary.

### I. Stem Cell Media

PPRF-msc6 is a defined serum-free growth medium developed for the isolation and expansion of hMSCs from bone marrow. PPRF-msc6 is prepared by adding growth and attachment factors, proteins, lipids, hormones, vitamins, pH buffers and nutrients to commercially available basal media, as described below. hMSCs isolated and expanded using PPRF-msc6 express high levels (>99%) of CD13, CD29, CD44, CD73, CD90, CD105, CD166 and HLA-ABC and are negative for CD14, CD19, CD34, CD45, and HLA-DR. These cells retain their multipotency such as adipogenic and osteogenic differentiation potential. Furthermore, PPRF-msc6 medium can also support the isolation and expansion of hMSCs from other sources such as adipose tissue.

A. Components

Basal Media/Nutrients. Basal media will comprise, in one example, Dulbecco's modified Eagle's medium (DMEM). A 5× DMEM stock solution is prepared by dissolving one packet of powdered DMEM (Gibco, 12100) in 200 mL of cell culture grade water. The reconstituted DMEM is then filtered through a 0.22 μm filter using a sterile Pyrex glass bottle and stored at 4° C. for a maximum period of two months.

Nutrients can be provided by Ham's F12 Nutrient Mixture (F12) or other media mixtures. A 10× F12 stock solution is prepared by dissolving one packet of powdered F 12 (Gibco, 21700) in 100 mL of cell culture grade water. The reconstituted F12 is then filtered through a 0.22 μm filter using a sterile Pyrex glass bottle and stored at 4° C. for a maximum period of two months.

Glutamine. A 200 mM sterile glutamine solution (Gibco, 25030) is purchased and stored at −20° C. in 5.0 mL aliquots. Prior to use, an aliquot is thawed in a 37° C. water bath, and then agitated to entirely dissolve the glutamine precipitated during the freeze-thaw process. The reminder is discarded.

Sodium Bicarbonate. A 7.5% sodium bicarbonate stock solution is prepared by dissolving 7.5 g of sodium bicarbonate (Sigma, S5761) into 100 mL of cell culture grade water. The stock solution is then filtered through a 0.22 μm filter and stored at −20° C. in 20 mL aliquots. Prior to use, an aliquot is thawed in a 37° C. water bath, and then agitated to completely dissolve the sodium bicarbonate precipitated during the freeze-thaw process. The remainder is placed at 4° C. for future use.

Hepes. A 1.0 M Hepes stock solution is prepared by dissolving 23.8 g of Hepes (Sigma, H4034) into 80 mL of cell culture grade water. Then, more water is added to the dissolved solution of Hepes until a total volume of 100 mL is reached. The stock solution is filtered through a 0.22 μm filter, aliquoted and stored at 4° C.

Lipid Concentrate. A chemically defined lipid concentrate (Gibco, 11905) is aliquoted into small Eppendorf tubes such that there is very little headspace (airspace) between the liquid and the cap to minimize fatty acid oxidation, and stored at 4° C. in the dark. Prior to use, an aliquot is agitated to dissolve the fatty acids precipitated during storage. The reminder is discarded.

Insulin. A 10 g/L insulin stock solution is prepared by adding 1.0 g of insulin from bovine pancreas (Sigma, 15500) into 100 mL of 0.1 N HCl and vortexing to dissolve the insulin. The stock solution is filtered, aliquoted and stored at −20° C.

Transferrin. A 10 g/L transferrin stock solution is prepared by dissolving 1.0 g of human apo-Transferrin (Sigma, T2252) into 100 mL of cell culture grade water. The stock solution is filtered, aliquoted and stored at −20° C. Synthetic recombinant alternative to native transferrin has been produced by InVitria (Fort Collins, Colo.). Moreover, the manufacturer reports that this recombinant protein performs equal to, or better than those native proteins in the growth of many mammalian cell types. Thus, the inventors contemplated the recombinant alternative to transferrin.

Putrescine. A 10 g/L putrescine stock solution is prepared by dissolving 0.5 g of putrescine dihydrochloride (Sigma, P7505) into 50 mL of cell culture grade water. The stock solution is filtered, aliquoted and stored at −20° C.

Progesterone. A 20 mg/L progesterone stock solution is prepared by adding 1.0 mL of 95%-100% sterile ethanol into 1.0 mg of progesterone (Sigma, P6149), gently swirling to dissolve, and then adding 49 mL of sterile cell culture grade water. The stock solution is filtered, aliquoted and stored at −20° C.

Basic Fibroblast Growth Factor (bFGF). A 20 mg/L stock solution of bFGF is prepared by reconstituting 25 μg of lyophilized recombinant human bFGF (R&D Systems, 233-FB) in 1.25 mL of sterile PBS containing 1.0 mg/mL human serum albumin and 4 mM HCl. The bFGF stock solution is aliquoted into 50 μL aliquots and stored at −80° C. Prior to use, an aliquot is thawed, and the remainder is placed at 4° C. for a maximum period of one month.

Transforming Growth Factor β1 (TGF-β1). A 10 mg/L stock solution of TGF-β1 is prepared by reconstituting 10 μg of lyophilized recombinant human TGF-β1 (R&D Systems, 240-B) in 1.0 mL of sterile PBS containing 1.0 mg/mL human serum albumin and 4 mM HCl. The TGF-β1 stock solution is aliquoted into 50 μL aliquots and stored at −20° C. Prior to use, an aliquot is thawed, and the remainder is placed at 4° C. for a maximum period of one month.

Hydrocortisone solution. A 50 μM sterile hydrocortisone solution (Sigma, H6909) is purchased and stored at −20° C. in 0.5-1.0 mL aliquots. When needed, an aliquot is thawed, and the reminder is discarded.

Ascorbic Acid. A 50 mg/mL stock solution of ascorbic acid-2-phosphate is prepared by dissolving 572.2 mg of L-ascorbic acid-2-phosphate sesquimagnesium salt (Sigma, A8960) in 10 mL of cell culture grade water. The stock solution is filtered, aliquoted and stored at −20° C.

Serum Albumin. Sterile human serum albumin in normal saline (100 mg/mL; InVitroCare, 2101) is purchased and stored at 4° C. Synthetic recombinant alternative to native human serum albumin has been produced by InVitria (Fort Collins, Colo.). Moreover, the manufacturer reports that this recombinant protein performs equal to, or better than native proteins in the growth of many mammalian cell types. Thus, the inventors contemplated this recombinant alternative to albumin.

Fetuin. Fetuin, a major plasma glycoprotein, has been identified to have various functions in cell culture, such as inhibiting trypsin activity and promoting cell attachment, growth, and differentiation, and thus recognized as an important supplement to serum-free media for a variety of cell types (reviewed in Nie, 1992). Fetuin from fetal bovine serum has been widely used as a supplement in cell culture media, while its homologue has also found in other species including human. In particular, fetuin was a requirement for serum-free primary culture of some types of cells such as more fibroblast and epithelial cells (Wang and Haslam, 1994). Fetuin can be prepared from fetal bovine serum according to three different methods by Pedersen, Deutsch, and Spiro. In the inventors' study, they used a commercial source of Pedersen fetuin, which is relatively less pure than the others, associating with several trace components. The mechanism of fetuin's growth-promoting activity is unknown. Although the activity on some cell lines were seemed to be due to its contaminant(s) (Rizzino and Sato, 1978), it was also demonstrated that fetuin itself appears to possess the action (Florini and Roberts, 1979). A powdered form of cell culture tested fetuin from fetal calf serum (Sigma, F3385) is purchased and stored at 4° C.

There are commercially available recombinant and native human fetuin produced by several manufacturers. However, all of these products are very expensive and provided at very low amounts for immunoassays (Table 1). The concentration of fetal calf fetuin present in PPRF-msc6 for the optimal growth of hMSCs is 1.0 g/L, and thus the currently available human fetuin products are may prove commercially impractical for hMSC cell culture.

TABLE 1
Commercially available recombinant or native human fetuin.

| | | | Size | Price |
|---|---|---|---|---|
| Source | Supplier | Cat # | (μg) | (USD) |
| Native | BioVendor | RD172037100 | 100 | $250 |
| Recombinant | R&D Systems | 1184-PI-050 | 50 | $315 |
| Native | Calbiochem | 362199 | 1,000 | $210 |
| Native | US Biological | F4102-20 | 50 | $315 |
| Native | ProSpec-Tany | PRO-418 | 1,000 | $2,400 |
| | TechnoGene LTD | | | |

Alternatively, through an extensive literature review, the inventors recently found an interesting result observed by Salomon and colleagues in 1982, which we think could represent a possible solution for the replacement of fetal calf fetuin with a human-sourced protein. Salomon et al. (1982) demonstrated that crude bovine fetuin (Pedersen fetuin) was required for maintaining the growth of mouse embryonal carcinoma cells and rat mammary epithelial cells in serum-free conditions. This requirement was not able to be replaced by purified fetuin preparations (i.e., Deutsch fetuin and Spiro fetuin). However, a growth-promoting protein, named embryonin, isolated from the crude Pedersen fetuin preparations was able to stimulate the growth of both cell types aforementioned in the absence of serum. The level of growth promotion by purified embryonin at 2.0 to 4.0 μg/mL was equivalent to that achieved with 0.5 to 1.0 mg/mL of the crude Pedersen fetuin preparation. This indicates that the actual growth-promoting activity of fetuin is likely due to one of its associated components, embryonin, rather than fetuin itself. More importantly in clinical viewpoint, the activity of the purified embryonin could be replaced by human α₂-macroglubulin, which has shown to be very similar to embryonin in many aspects. It was demonstrated that human α₂-macroglubulin used over the same concentration range as embryonin provided a comparable growth stimulation for mouse embryonal carcinoma cells in the absence of serum (Salomon et al., 1982).

Sigma supplies α₂-macroglubulin from human plasma (lyophilized powder, ≧98%) at various amounts, e.g., 10 mg (Cat M6159). Therefore, in view of the study by Salomon et al. (1982) in which ˜4.0 mg/L of α₂-macroglubulin resulted in an equivalent growth of mouse embryonal carcinoma cells achieved with 1.0 g/L of fetuin, 4.0 mg of α₂-macroglubulin can be used to replace 1.0 g of fetuin included in 1.0 L of PPRF-msc6. Considered together, the inventors are currently testing α₂-macroglubulin for its ability to support the isolation and expansion of hMSCs.

B. Medium Preparation

The following protocol was used to make 1.0 L of PPRF-msc6 medium. Using graduated cylinders, 764.417 mL of cell culture grade water was measured and placed into a sterile 1000 mL glass bottle. Using a pipette, the following supplements were added into the bottle.

a. 100 mL of 5× DMEM

b. 50 mL of 10× F12

c. 7.5 mL of 200 mM glutamine solution

d. 23 mL of 7.5% sodium bicarbonate stock solution

e. 4.9 mL of 1 M hepes stock solution

f. 40 mL of 100 g/L serum albumin

g. 1.0 mL of lipid concentrate

Using a pipette, the following supplements were thawed and added into the bottle. Unused components remaining after making medium were discarded.

h. 2.3 mL of insulin

i. 2.5 mL of apo-transferrin

j. 0.9 mL of putrescine

k. 0.283 mL of progesterone

l. 100 μL of bFGF

m. 100 μL of TGF-β1

n. 2.0 mL of hydrocortisone

1.0 g of fetuin was weighed out, added into the bottle, and allowed to dissolve at room temperature. The prepared medium was filtered through a 0.22 μm filter into a second sterile Pyrex glass bottle.

The medium, i.e., PPRF-msc6 medium lacking ascorbic acid, was stored at 4° C. in the dark for a maximum period of one month.

When needed, an appropriate amount of this medium was transferred to a separate vessel and incubated at 37° C. and 5% CO₂ for 2 hours prior to use. Finally, using a pipette, an ascorbic acid stock solution was thawed and added at a rate of 1 mL per L of medium into the preheated medium in a sterile manner.

### II. Cells and Isolation

A. Cell Types

The present invention may be utilized with a variety of different cell types that are generally referred to as stem cells, including mesenchymal stem cells or MSCs. Specific examples of MSCs are those obtained from human bone marrow and adipose tissue.

Stem cells may be described as those cells which exhibit one or more of the following characteristics (i) they are plastic-adherent when maintained in culture, (ii) they express high levels (≧95%) of CD105, CD73 and CD90 and lack expression of CD45, CD34, CD14 or CD11b, CD79a or CD19 and HLA-DR surface molecules, and (iii) they are able to differentiate into multiple cell types such as osteoblasts and adipocytes under specific in vitro differentiation conditions (Dominici et al., 2006).

B. Isolation Procedures

Stem cells may be isolated as follows. For bone marrow-derived human mesenchymal stem cells (BM-hMSCs), frozen human bone marrow mononuclear cells (BM MNCs) were purchased from Lonza (formerly Cambrex Biosciences, Walkersville, Md.). Lonza obtained the cells by withdrawing human bone marrow from bilateral punctures of the posterior iliac crest of the pelvic bone of healthy donors using syringes containing heparin sodium (1,000 units of heparin per ml bone marrow). The bone marrow was then diluted to 5-10 million nucleated cells per mL in Hank's Buffered Salt Solution (HBSS). Cells were layered over Ficoll-Paque (Amersham Pharmacia, cat #17-1440-03) and centrifuged at 400×g for 30 minutes at room temperature. The mononuclear cell layer was removed and washed twice in HBSS. Finally, the BM MNCs were cyropreserved in a cell cryopreservation medium comprising 86.5% IMDM, 7.5% DMSO, 4% Human Serum Albumin and 2% hydroxy-ethyl-starch. Upon arrival, we at PPRF stored the frozen cells in liquid nitrogen for later experimental use. When needed, the cryopreserved BM MNCs were thawed, washed and plated into tissue culture flasks or plates according to the manufacturer's instructions with a minor modification. Briefly, the frozen BM MNCs were quickly thawed in a 37° C. water-bath, diluted with pre-warmed wash solution comprising a cytokine-free version of PPRF-msc6 medium and 20 U/mL of DNase I (Sigma-Aldrich, St. Louis, Mo.), and centrifuged at 200×g for 15 minutes. The BM MNCs were rinsed twice with the cytokine-free version of PPRF-msc6 medium, counted, and plated at 150,000 cells/cm² into tissue culture flasks containing experimental growth media (i.e., control 10% FBS DMEM from Lonza versus the defined serum-free PPRF-msc6 medium). The tissue culture flasks were coated with 0.1% gelatin from bovine skin (Sigma-Aldrich) prior to use. Preliminary experiments showed that a surface coated with gelatin, or another protein such as fibronectin, encouraged cell adherence and growth thereby facilitating rapid isolation of MSCs in serum-free conditions without affecting their differentiation potential. The plated cells were cultured in a humidified incubator at 37° C. and 5% CO₂. After 48 hours, non-adherent cells were removed and fresh medium was added. Thereafter, 50% of medium was replenished every other day. When well-developed colonies appeared (normally on days 10-13), the medium was removed and the adherent cells were trypsinized with 0.25% trypsin and 1 mM EDTA (Invitrogen, Grand Island, N.Y.) for 3-5 minutes at 37° C. For the control serum-based cultures, the adherent cells were rinsed twice with Dulbecco's Phosphate-Buffered Saline (PBS, Invitrogen) prior to the trypsinization. For both serum-free and serum-based cultures, the trypsin was neutralized using the control 10% FBS DMEM, and the cells were subsequently pelleted by centrifugation at 300×g for 10 min and resuspended in warm culture medium. The cells were rinsed by the same centrifugation/resuspension protocol. For the control serum-based cultures, the additional wash step was not necessary.

Primary adipose tissue-derived human mesenchymal stem cells (AT-hMSCs) isolated exzymatically under serum-free conditions from abdominal subcutaneous adipose tissues were plated into either the defined serum-free PPRF-msc6 medium or a control serum-supplemented medium from Lonza (i.e., 10% FBS DMEM) in fibronectin-coated tissue culture flasks at a density of 2,000 cells/cm². The cells were allowed to grow in a humidified incubator at 37° C. and 5% CO₂. After 48 hours, non-adherent cells were removed and fresh medium was added. Thereafter, 50% of the medium was replenished every other day. When the adherent cells grew to form well-developed colonies or reach confluence, the medium was removed and the adherent cells were trypsinized with 0.25% trypsin and 1 mM EDTA for 3-5 min at 37° C. For the control serum-based cultures, the adherent cells were rinsed twice with PBS prior to the trypsinization. For both serum-free and serum-based cultures, the trypsin was neutralized using the control 10% FBS DMEM, and the cells were subsequently pelleted by centrifugation at 300×g for 10 min and resuspended in warm culture medium. The cells were rinsed by the same centrifugation/resuspension protocol. For the control serum-based cultures, the additional wash step was not necessary.

### III. Expansion Procedures

Stem cells may be expanded through multiple passages as follows. The hMSCs isolated from bone marrow or adipose tissue using the methods invented herein were counted and the viable cell density was determined using the trypan blue dye exclusion method. To subculture (or passage) cells, they were replated at a density of 5,000 cells/cm² into the culture medium that was used for the isolation of hMSCs in new tissue culture flasks coated with protein such as gelatin or fibronectin, and were allowed to grow in a humidified incubator at 37° C. and 5% CO₂. If necessary, 50% of medium was replenished every three days. On reaching subconfluence (˜90%), the medium was removed and the adherent cells were trypsinized with 0.25% trypsin and 1 mM EDTA for 3-5 min at 37° C. For the control serum-based cultures, the adherent cells were rinsed twice with PBS prior to the trypsinization. For both serum-free and serum-based cultures, the trypsin was neutralized using the control 10% FBS DMEM, and the cells were subsequently pelleted by centrifugation at 300×g for 10 min and resuspended in warm culture medium. The cells were rinsed by centrifugation at 300×g for 10 min and resuspended in warm culture medium. For the control serum-based cultures, the additional wash step was not necessary. The harvested cells were subcultured through multiple passages using the same passage protocol.

### IV. Examples

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Isolation and Serial Expansion of hMSCs from Bone Marrow Using PPRF-msc6 Medium

For this example, as well as Example 2, the constituent of PPRF-msc6 medium was used for all primary and subsequent cultures described herein and is shown in Table 2. The isolation and subsequent serial expansion of hMSCs were performed in a humidified incubator at 37° C. and 5% CO₂ for all cultures described herein. The cultured cells were characterized as hMSCs by performing various assays such as CFU-F assay, flow cytometry analysis, and differentiation assays.

TABLE 2
Components of PPRF-msc6 medium for human
mesenchymal stem cell (hMSC) culture

| Component | Supplier & Cat # | Final Concentration | |
|---|---|---|---|
| DMEM | Gibco 12100 | 0.5 | x |
| F12 | Gibco 21700 | 0.5 | x |
| Glutamine | Gibco 25030 | 1.5 | mM |
| Sodium bicarbonate | Sigma S5761 | 1.725 | g/L |
| Hepes | Sigma H4034 | 4.9 mM (1.167 g/L) | |
| Serum albumin | InVitro Care 2101 | 4 | g/L |
| Lipid concentrate | Gibco 11905 | 1 | x |
| Insulin | Sigma I5500 | 23 | mg/L |
| apo-Transferrin | Sigma T2252 | 25 | mg/L |
| Putrescine | Sigma P7505 | 9.0 | mg/L |
| Progesterone | Sigma P6149 | 5.66 | μg/L |
| Fetuin | Sigma F3385 | 1.0 | g/L |
| recombinant bFGF | R&D systems 230-FB | 2.0 | μg/L |
| recombinant TGF-β1 | R&D systems 240-B | 1.0 | μg/L |
| Hydrocortisone solution | Sigma H6909 | 100 | nM |
| Ascorbic acid-2-phosphate | Sigma A8960 | 50 | mg/L |

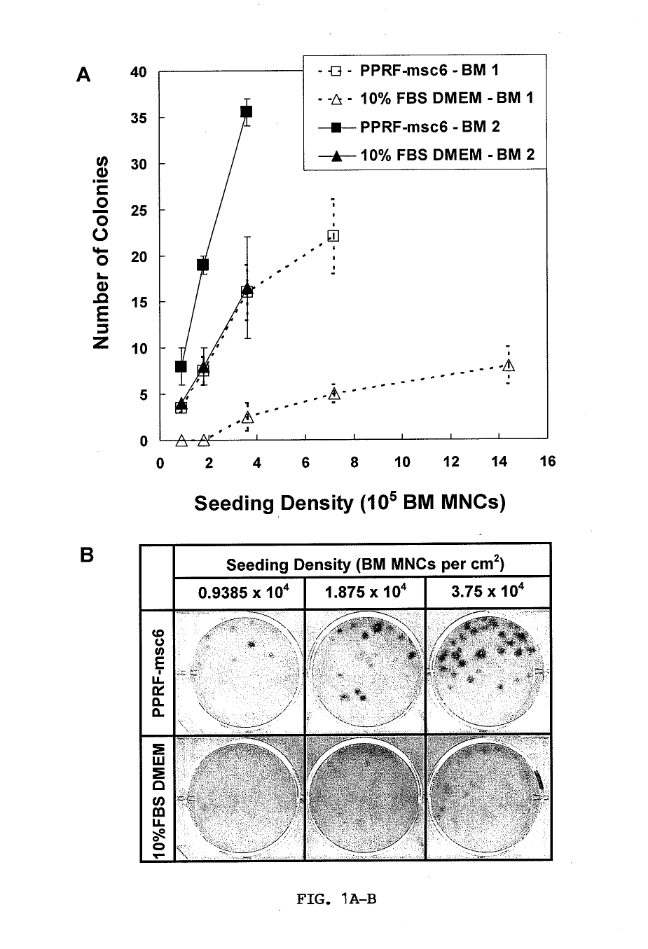

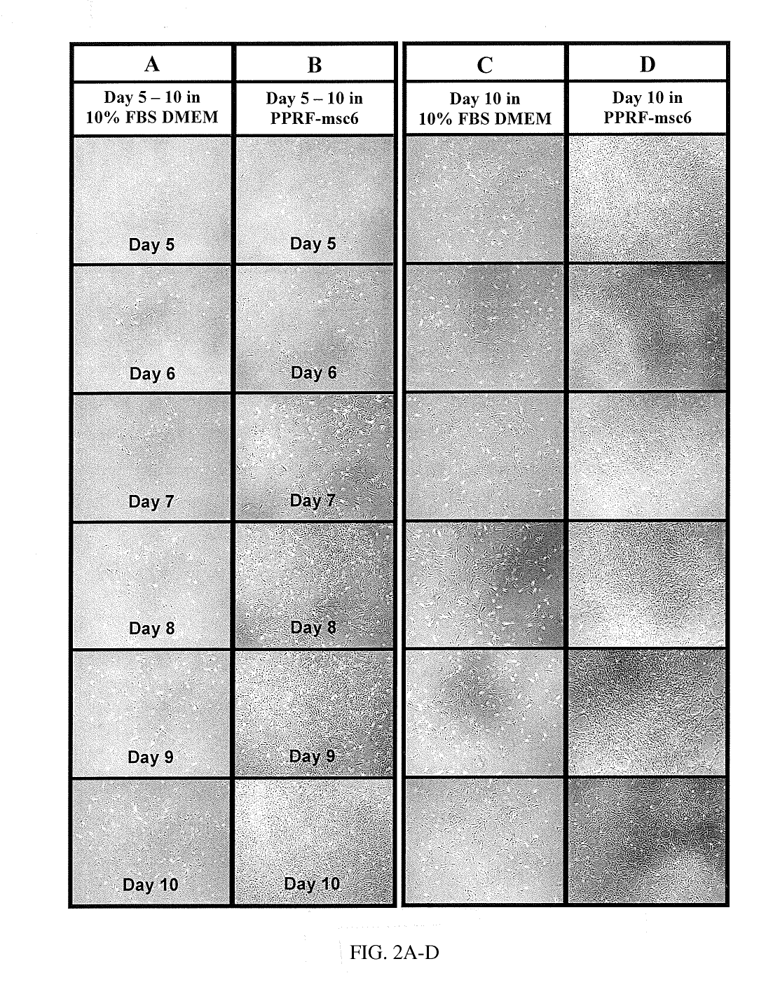

PPRF-msc6 was compared to FBS-supplemented medium from Lonza (10% FBS DMEM) for the isolation of hMSCs from bone marrow (BM-hMSCs). As described in the methods invented herein, cryopreserved BM MNCs were thawed, washed and plated into protein-coated tissue culture flasks containing growth medium. As shown in FIG. 1A, PPRF-msc6 yielded significantly higher numbers of hMSC colonies from bone marrow than FBS-supplemented medium for the same culture period. Moreover, the formed CFU-F colonies in PPRF-msc6 had a larger size (FIG. 1B). FIGS. 2A and 2B show the faster formation of CFU-F colonies during days 5-10 in PPRF-msc6 (FIG. 2B) compared to 10% FBS DMEM (FIG. 2A), whereas FIGS. 2C and 2D represents various individual colonies on day 10, showing bigger and denser colonies were formed in PPRF-msc6.

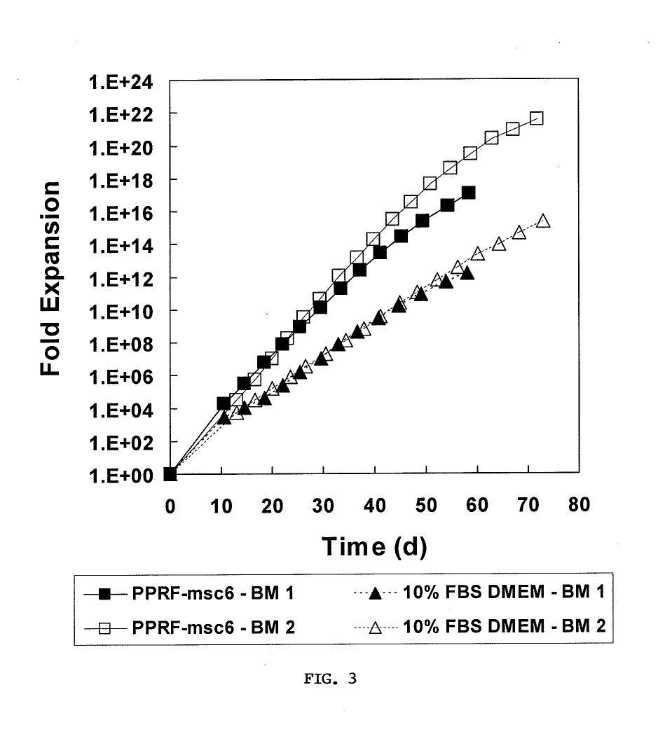

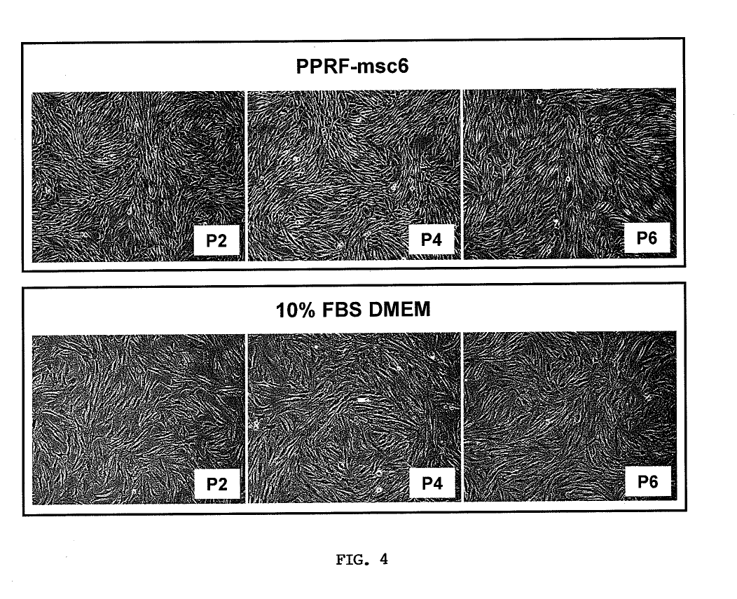

Isolated BM-hMSCs were expanded through multiple passages using PPRF-msc6 versus 10% FBS DMEM as described in the methods invented herein. FIG. 3 presents the comparison of growth kinetics of these cells in each of these media, and shows that PPRF-msc6 supported more rapid cell expansion than FBS-supplemented medium. Specifically, on the assumption that the frequency of hMSCs in the population of BM MNCs was 0.001% as described in other publications (Castro-Malaspina et al., 1984; Pittenger et al., 1999), PPRF-msc6 resulted in a 7.29×10⁹ cell-fold expansion within 30 days in culture when the plating density for primary and subsequent cultures was 150,000 BM MNCs (corresponding to 1.5 hMSCs) per cm² and 5,000 hMSCs per cm², respectively. This was significantly higher than the 1.17×10⁶ cell-fold expansion achieved over the same time period using the FBS-supplemented medium. FIG. 4 shows morphologies of BM-hMSCs expanded over the same time period in both media at passage levels 2, 4, and 6. Cells grown in PPRF-msc6 were smaller in size but expanded more rapidly compared to cells growing in FBS-supplemented medium. Importantly, there was no significant difference in morphology and growth rate between the passage levels observed.

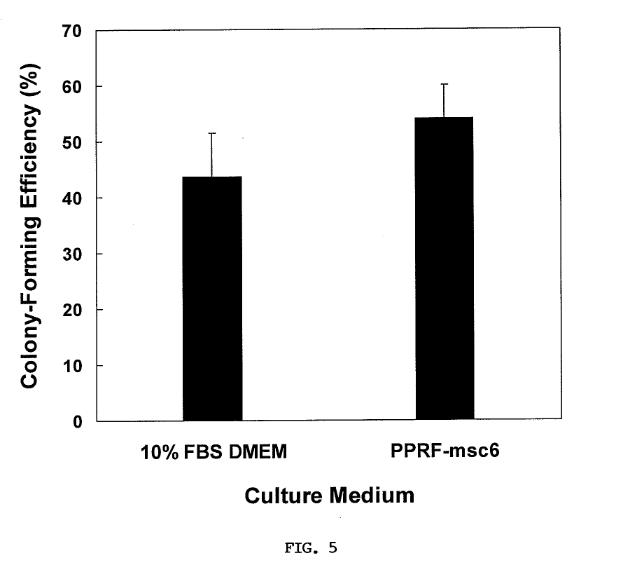

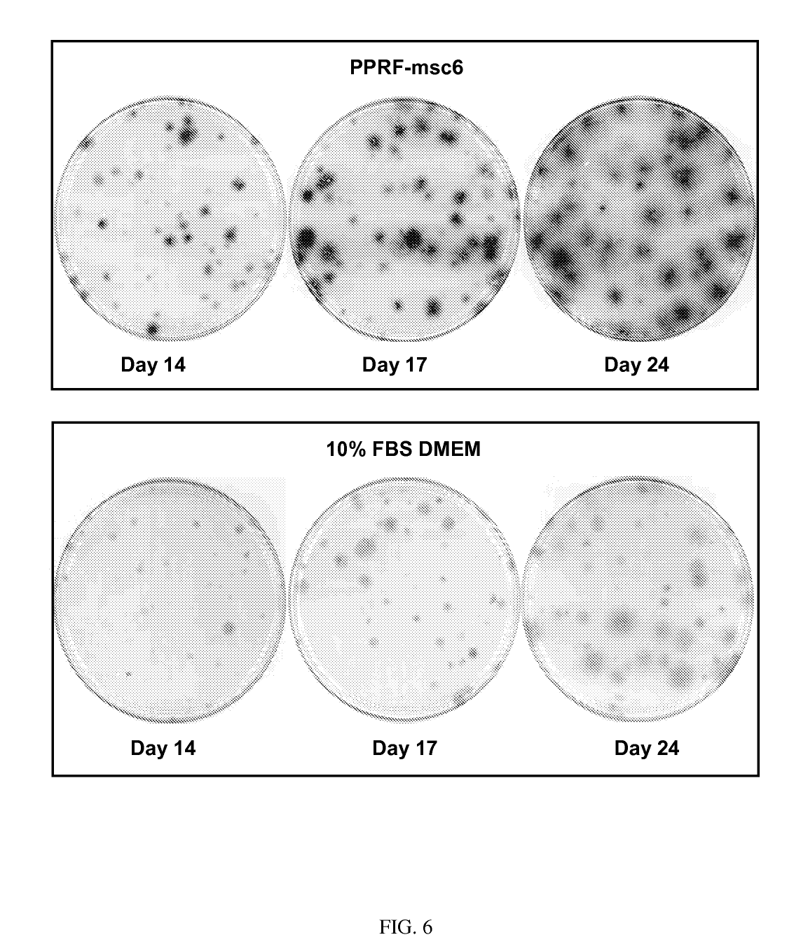

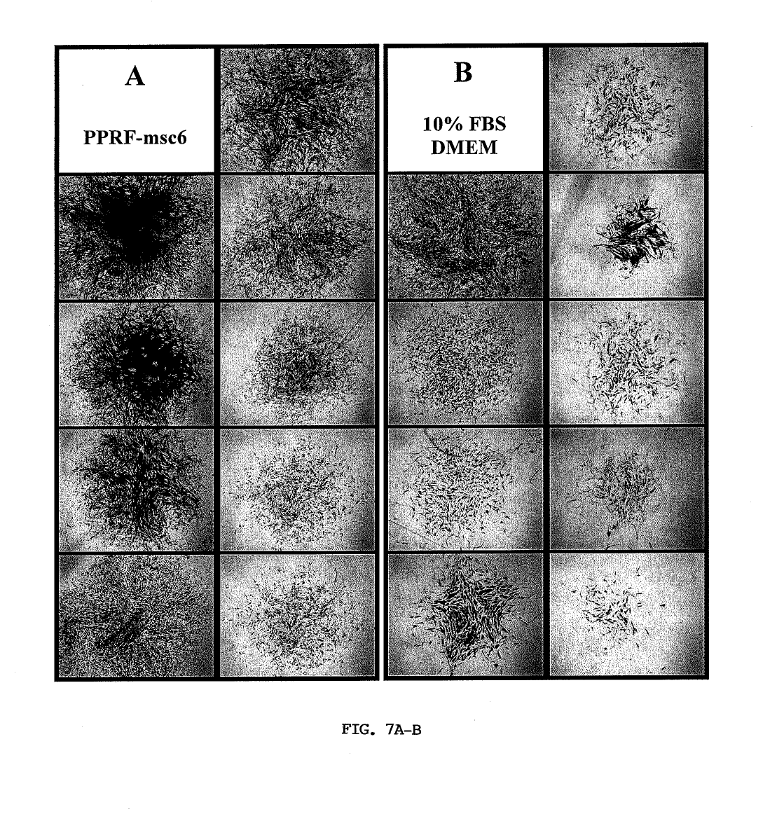

The frequency, size and density of CFU-F colonies derived from BM-hMSCs cultured in the two different media were also compared. For the CFU-F assay, BM MSCs at passage 4 grown in either PPRF-msc6 or 10% FBS DMEM were harvested, and seeded at 100 cells/dish in gelatin-coated 60 cm² dishes using the medium in which they had been expanded. The resulting colonies were stained with 0.5% crystal violet in methanol for visualization on multiple days. As shown in FIG. 5, it was determined that the CFU-F frequency of the cells expanded in PPRF-msc6 and 10% FBS DMEM was 54.00±6.00% and 43.75±7.80% (n=4), respectively, indicating that higher or at least comparable numbers of colonies can be obtained in PPRF-msc6 medium. FIG. 6 shows colonies at days 14, 17 and 24 derived from BM-hMSCs, whereas FIGS. 7A-B represent various kinds of individual colonies on day 14 during the CFU-F assay, showing that the colonies in PPRF-msc6 were generally bigger and denser. These results observed from CFU-F assay further supported that BM-hMSCs isolated and expanded in PPRF-msc6 had a higher proliferative potential compared to those cultured in FBS-supplemented medium.

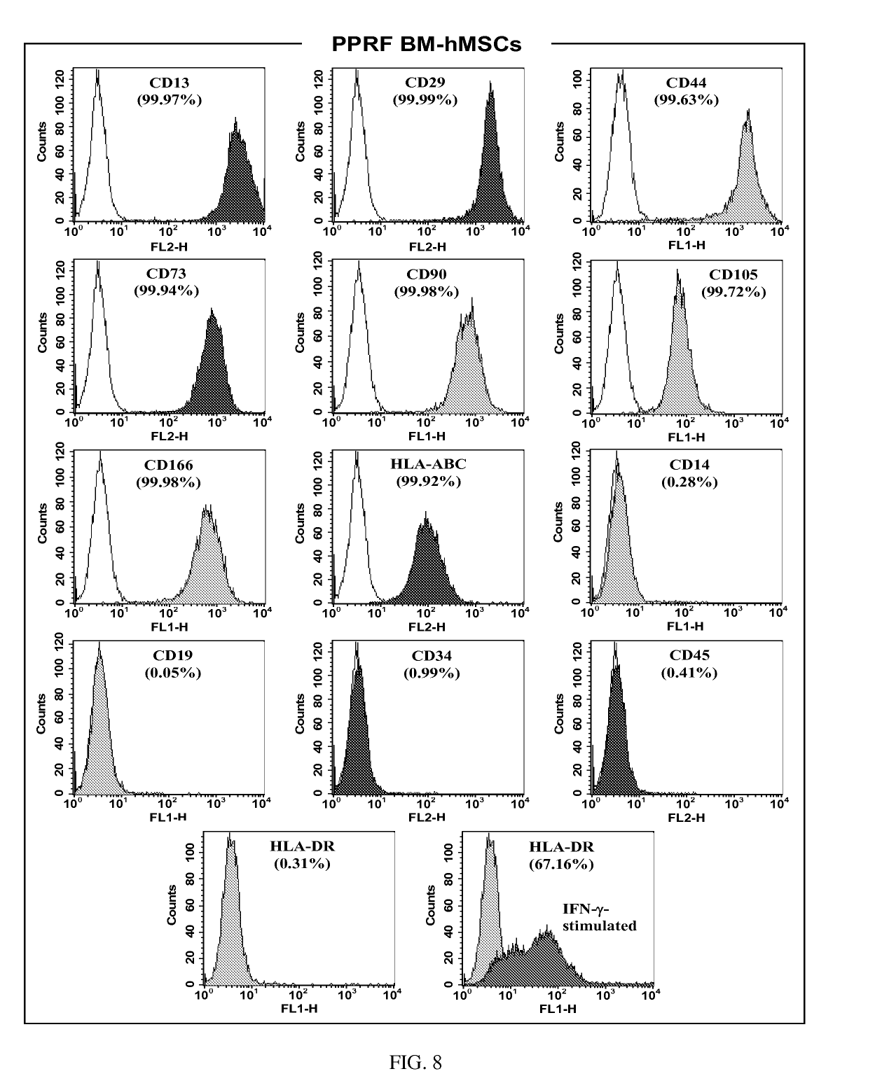

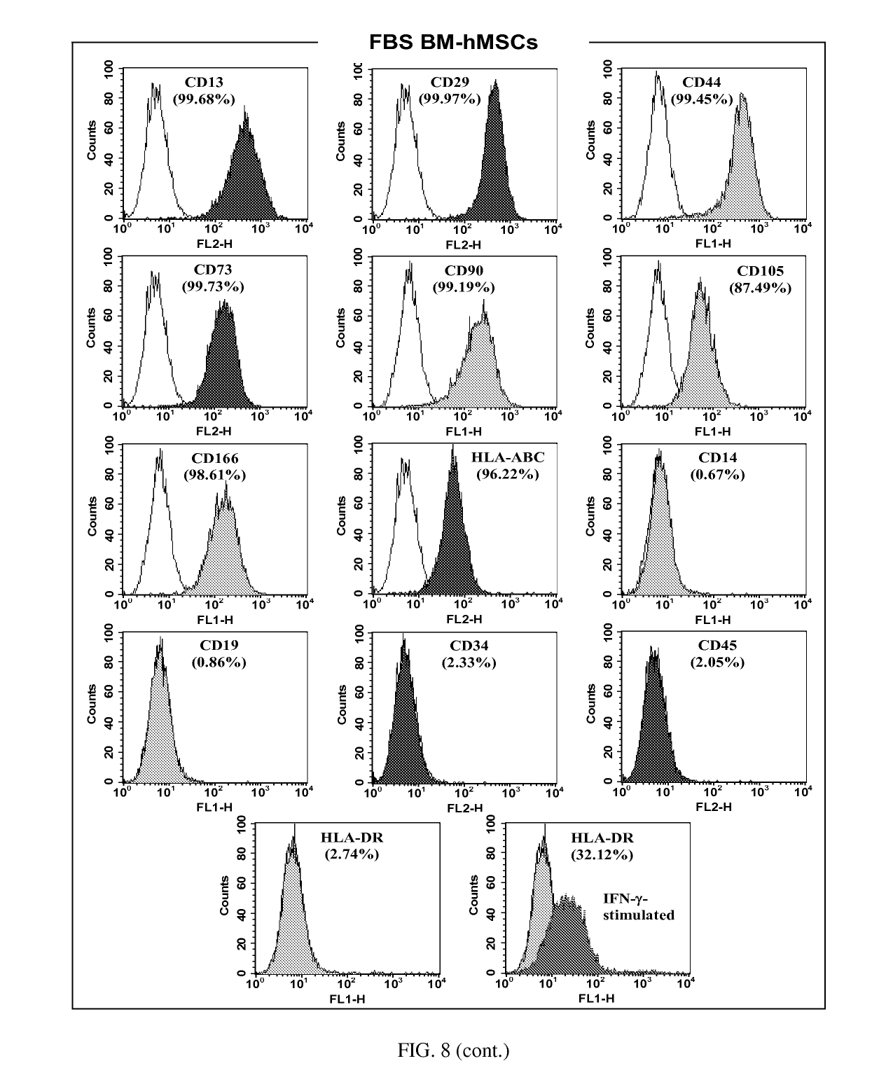

BM-hMSCs isolated and expanded under the different media were analyzed for their surface antigen expression at passage 3. As described in FIG. 8, cells cultured using PPRF-msc6 expressed high levels (>99%) of CD13, CD29, CD44, CD73, CD90, CD105, CD166 and HLA-ABC and were negative for CD14, CD19, CD34, CD45, and HLA-DR, satisfying the criteria defined for typical MSC-specific surface antigen expression in the literature (Dominici et al., 2006). It is also well known that hMSCs do not express HLA-DR surface molecules, but they do when stimulated by interferon (IFN)-γ (Dominici et al., 2006). It was demonstrated in our experiment that, in the presence of human recombinant IFN-γ (Invitrogen), HLA-DR molecules were upregulated on both cell populations (FIG. 8).

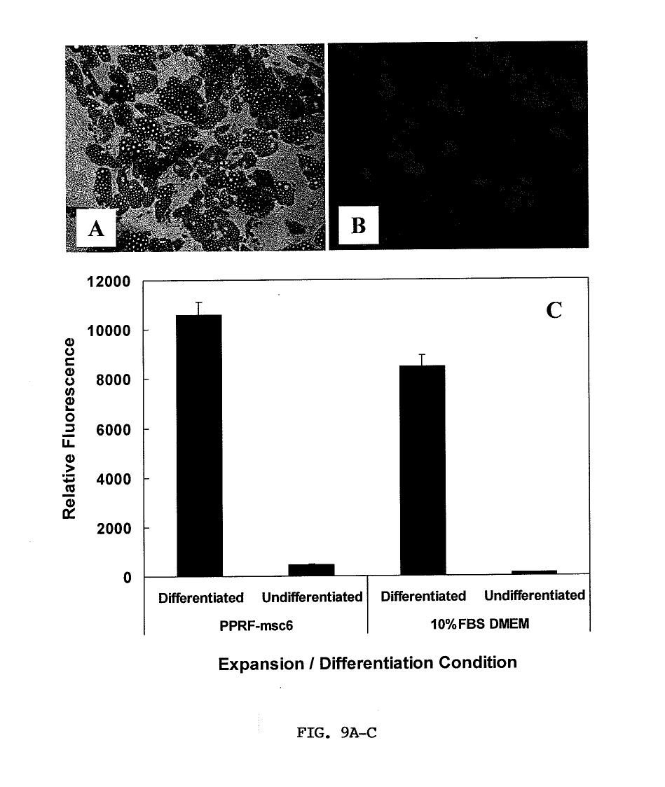

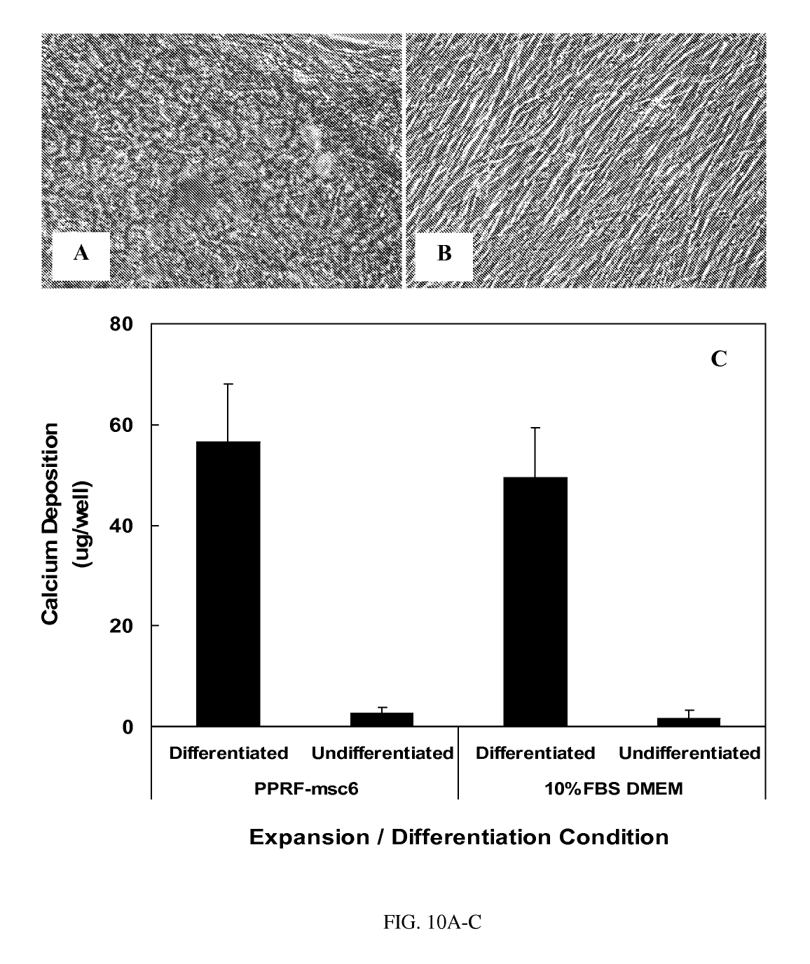

BM-hMSCs cultured using the two different media were also compared for their degree of multipotency in vitro. For these experiments, BM-hMSCs isolated and expanded for 27 days (at passage 4) in either PPRF-msc6 or 10% FBS DMEM were harvested and induced for adipogenesis and osteogenesis in tissue culture six-well plates using standard differentiation protocols. FIGS. 9A and 9B represent the adipogenic culture of BM-hMSCs, previously cultured in PPRF-msc6, which was stained with AdipoRed assay reagent (Lonza) after 3 weeks of induction in bright and fluorescent field, respectively. The accumulation of intracellular lipids stained was also quantitatively compared by measuring relative fluorescence as shown in FIG. 9C. This result indicates that cells cultured in PPRF-msc6 retained a higher or at least comparable degree of in vitro adipogenic potential compared to those cultured under the FBS-supplemented medium. FIGS. 10A and 10B show the osteogenic induced and non-induced cultures, respectively, of BM-hMSCs previously isolated and expanded in PPRF-msc6. These cultures were stained with Alizarin Red S dye after 2 weeks of induction to detect calcium mineralization. The degrees of calcium deposition in the induced cultures along with non-induced cultures were quantitatively compared using a spectrophotometer as shown in FIG. 10C, showing that cells cultured in PPRF-msc6 retained a comparable degree of in vitro osteogenic potential compared to those cultured under the FBS-supplemented medium.

### Example 2

### Isolation and Serial Expansion of hMSCs from Adipose Tissue Using PPRF-msc6 Medium

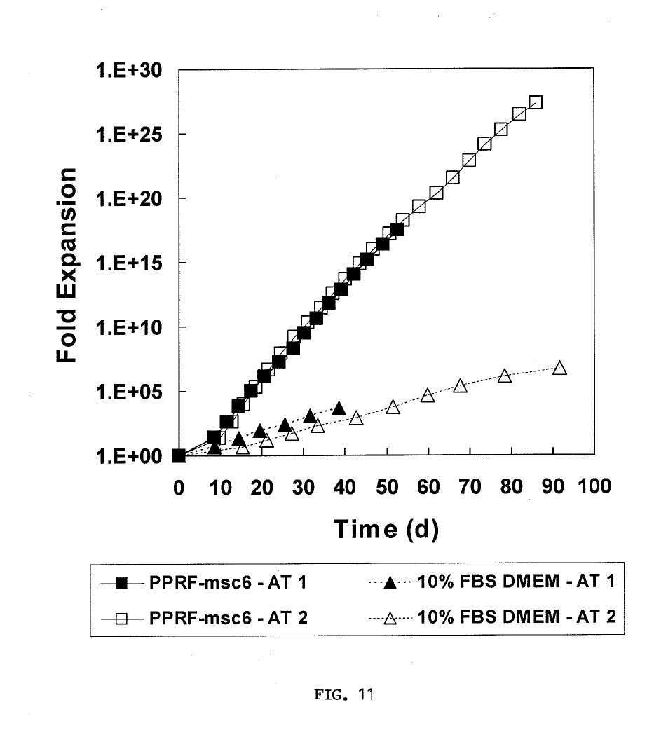

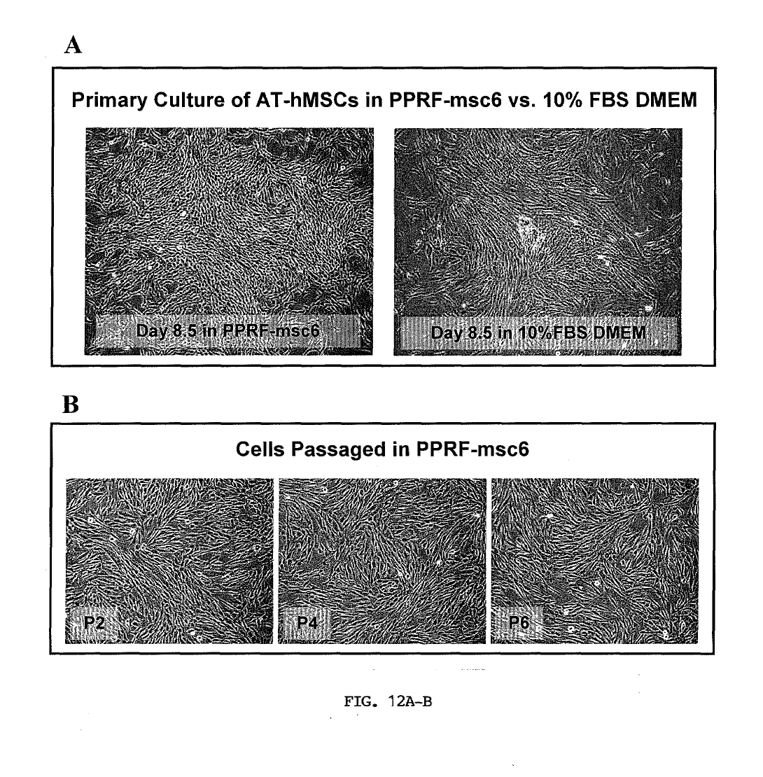

The isolation of hMSCs from adipose tissue (AT-hMSCs) and their serial expansion were compared using PPRF-msc6 and FBS-supplemented medium from Lonza (10% FBS DMEM). As described in the methods invented herein, a population of cells derived from human abdominal subcutaneous adipose tissue was plated at a density of 2,000 cells/cm² into protein-coated tissue culture flasks containing growth medium. After 48 hours, non-adherent cells were removed and fresh medium was added. Thereafter, 50% of the medium was replenished every other day. When well-developed colonies appeared in the cultures, the adherent cells were harvested by trypsinization, counted, and replated into new flasks at a density of 5,000 cells/cm². The serial passaging of cells were performed using the same passage protocols. FIG. 11, which presents the growth kinetics of primary and subsequent cultures of AT-hMSCs in both PPRF-msc6 and 10% FBS DMEM, shows that the cells were isolated and expanded in PPRF-msc6 at a higher rate. FIG. 12A shows morphologies of AT-hMSCs in the primary culture. Cells cultured in PPRF-msc6 formed colonies more rapidly and densely, which led to a higher yield in the isolation of AT-hMSCs. FIG. 12B presents morphologies of AT-hMSCs expanded in PPRF-msc6 at passage levels 2, 4, and 6, indicating that there was no significant change in morphology and growth rate over time.

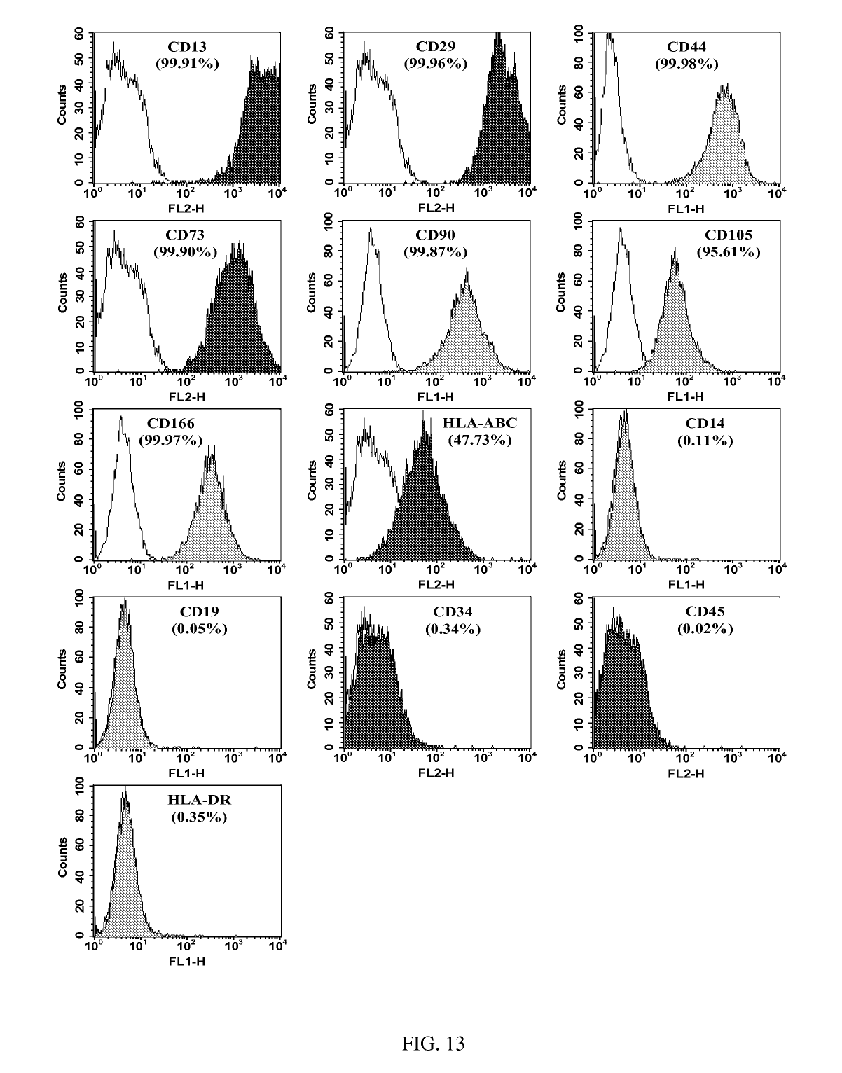

AT-hMSCs isolated and expanded in PPRF-msc6 were analyzed for their surface antigen expression pattern at passage 5. As described in FIG. 13, AT-hMSCs cultured using PPRF-msc6 expressed high levels of CD13, CD29, CD44, CD73, CD90, CD105 and CD166 and were negative for CD14, CD19, CD34, CD45, and HLA-DR. Interestingly, the expression level (47.73%) of HLA-ABC on AT-hMSCs cultured in PPRF-msc6 was much lower, when compared to the counterpart from human bone marrow (see FIG. 8).

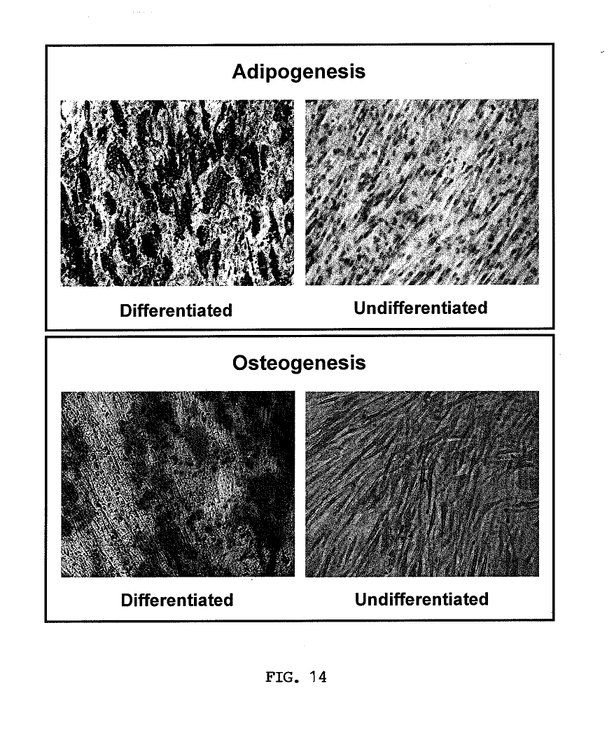

AT-hMSCs cultured in PPRF-msc6 were also characterized for their multipotency in vitro. For these experiments, AT-hMSCs isolated and expanded up to passage 5 in PPRF-msc6 were harvested and induced for adipogenesis and osteogenesis in tissue culture six-well plates using standard differentiation protocols. FIG. 14 presents the induced cultures along with control non-induced cultures, showing that AT-hMSCs isolated and expanded under PPRF-msc6 were multipotent. The adipogenic cultures were stained with Oil Red O stain on day 18 of induction to detect the accumulation of intracellular lipids, whereas the osteogenic culture were stained with Alizarin Red S dye after 3 weeks of induction to visualize calcium deposition.

### Example 3

### Isolation and Serial Expansion of hMSCs from Adipose Tissue Using PPRF-msc6 Medium

The inventors have defined factors required for the isolation and subsequent expansion of tissue-specific hMSCs under new serum-free conditions. PPRF-msc6 represents the most well-defined serum-free formulation described in the literature to date, for (i) the successful isolation of hMSCs from primary cultures of bone marrow and other sources such as adipose tissue and (ii) the subsequent expansion of these isolated MSCs through multiple passages while maintaining high proliferation rates. Although all the ingredients are defined in terms of origin and purity, PPRF-msc6 is not considered to be a xeno-free medium as the insulin and fetuin used in this medium are purified from animal serum. It is well known that these purified components can be associated with traces of other serum constituents, which may affect cell growth. Nonetheless, the development of PPRF-msc6 represents a significant step forward as it is the most well-defined medium reported to date, and will facilitate the standardization of BM-hMSC production, and the subsequent implementation of these cells in clinical applications.

Ideal cell culture media should use synthetic recombinant proteins, if available, for their requirement of protein constituents, excluding animal- and/or human-derived products. Therefore, an alternative to the existing PPRF-msc6 would be to replace the native components in PPRF-msc6 with synthetic materials. In this regard, one or more of insulin, transferrin, albumin and fetuin would be replaced.

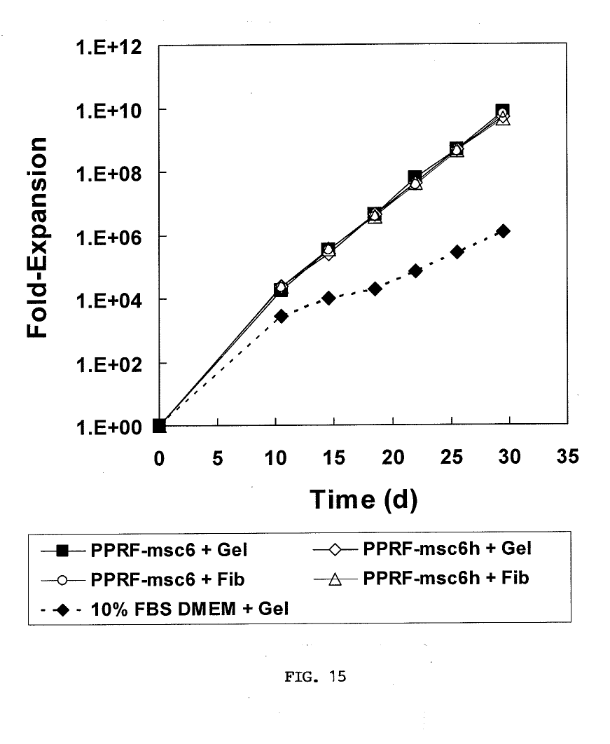

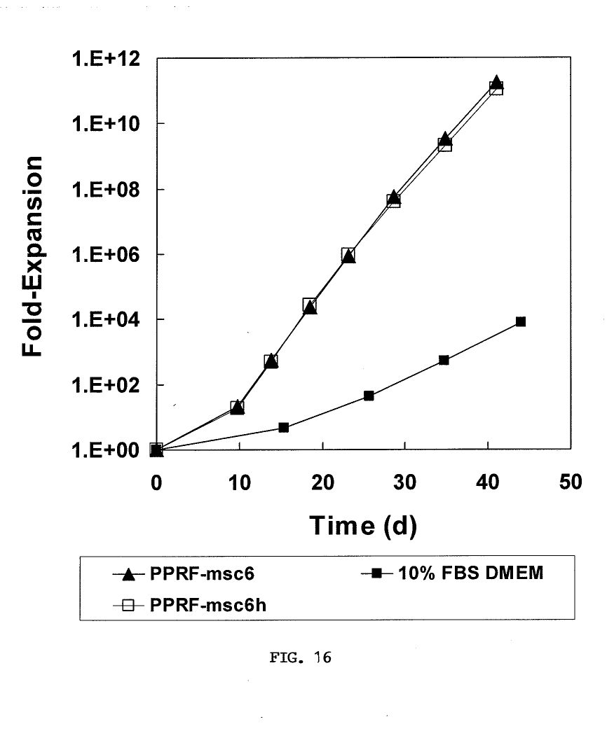

Insulin. The inventors have tested the effect of recombinant human (rh) insulin (Sigma, I9278) in comparison with bovine pancreas-derived insulin in PPRF-msc6 for the isolation and serial expansion of BM-hMSCs. PPRF-msc6 containing rh-insulin instead of bovine insulin is referred herein to as PPRF-msc6h, and both PPRF-msc6 and PPRF-msc6h contained identical concentration of their respective insulin. BM-hMSC growth in these media was evaluated under two different substrate conditions—i.e., on gelatin (bovine)-coated and fibronectin (human)-coated surface. It was demonstrated that both PPRF-msc6 and PPRF-msc6h supported the isolation and expansion of BM-hMSCs at a comparable rate, regardless of the coating material, and were superior in these respects to FBS-supplemented medium (FIG. 15). Moreover, BM-hMSCs cultured in PPRF-msc6 versus PPRF-msc6h displayed an identical phenotype (Table 3). PPRF-msc6h was also able to support the isolation and expansion of AT-hMSCs in a comparable manner with PPRF-msc6 (FIG. 16). Considered together, it is expected that bovine insulin present in PPRF-msc6 can be successfully replaced with rh-insulin without impacting hMSC growth.

TABLE 3
Comparison of surface antigen expression levels of BM-hMSCs
at passage 3 cultured in PPRF-msc6 + gelatin (bovine)-coated
substrate versus PPRF-msc6h^{a} + fibronectin (human)-coated
substrate by flow cytometry analysis

Culture Condition

| Surface Antigen | PPRF-msc6 + Gel | PPRF-msc6 + Fib |
|---|---|---|
| CD13 | 99.97 | 99.93 |
| CD29 | 100.00 | 100.00 |
| CD44 | 99.90 | 99.89 |
| CD73 | 99.89 | 99.78 |
| CD90 | 100.00 | 99.99 |
| CD105 | 99.82 | 99.99 |
| CD166 | 100.00 | 99.99 |
| HLA-ABC | 99.99 | 99.99 |
| CD14 | 0.26 | 0.18 |
| CD19 | 0.04 | 0.04 |
| CD34 | 1.87 | 1.68 |
| CD45 | 0.69 | 0.90 |
| HLA-DR | 0.29 | 0.15 |

^{a}PPRF-msc6h was prepared by replacing purified bovine insulin in PPRF-msc6 with recombinant human insulin
Abbreviations: BM-hMSCs, bone marrow-derived human mesenchymal stem cells; Gel, gelatin; Fib, fibronectin

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. References

The following references, to the extent that they provide exemplary procedural or other details supplementary to those set forth herein, are specifically incorporated herein by reference.
Capelli et al., Bone Marrow Transplant., 40:785-791, 2007
Caplan, J. Cell. Physiol., 213(2):341-347, 2007
Castro-Malaspina et al., Prog. Clin. Biol. Res. 1984
Dimarakis and Levicar, Stem Cells, 24:1407-1408, 2006
Dominici et al., Cytotherapy, 8(4):315-317, 2006
Doucet et al., J. Cell. Physiol., 205:228-236, 2005
Lange et al., J. Cell. Physiol., 213:18-26, 2007
Le Blanc et al., Transplantation, 84:1055-1059, 2007
Lennon et al., Exp. Cell. Res., 219(1):211-222, 1995.
Liu et al., Biochem. Eng. J, 33:1-9, 2007
Mannello and Tonti, Stem Cells, 25:1603-1609, 2007
Müller et al., Cytotherapy, 8:437-444, 2006
Parker et al., Cytotherapy, 9(7):637-646, 2007
Pittenger et al., Science, 284(5411):143-147, 1999
Shahdadfar et al., Stem Cells, 23(9):1357-1366, 2005
Stute et al., Exp. Hematol., 32:1212-1225, 2004
Tropel et al., Stem Cells, 24(12):2868-2876, 2007
U.S. Pat. No. 5,908,782
U.S. Patent Publication 2005/0265980 A1
U.S. Pat. No. 7,109,032 B2

What is claimed is:

1. A culture medium comprising, dissolved or dispersed in base culture medium and water, the following components:
(a) nutrients;
(b) glutamine;
(c) sodium bicarbonate;
(d) hepes;
(e) serum albumin;
(f) lipids;
(g) insulin;
(h) transferrin;
(i) putrescine;
(j) progesterone;
(k) fetuin or α₂-macroglubulin;
(l) hydrocortisone;
(m) ascorbic acid;
(n) bFGF; and
(o) TGF-β1.

2. The medium of claim 1, wherein the base culture medium is MCDB media series, CMRL medium-1066, Roswell Park Memorial Institute (RPMI) medium, alpha Modified Eagle's Medium (α-MEM), Dulbecco's Modified Eagle's Medium (DMEM) or Iscove's Modified Dulbecco's Medium (IMDM).

3. The medium of claim 2, wherein the base culture medium is Dulbecco's Modified Eagle's Medium

4. The medium of claim 1, wherein nutrients comprise Ham's F12 nutrient mixture.

5. The medium of claim 1, wherein sodium bicarbonate is present at about 0.1 g/L to about 4.0 g/L, hepes is present at about 1.0 mM to about 10 mM, serum albumin is present at about 0.1 g/L to about 10 g/L, fetuin is present at about 0.1 g/L to about 10 g/L, α₂-macroglubulin is present at about 0.4 mgL to about 40 mg/L, hydrocortisone is present at about 1.0 nM to about 1000 nM, ascorbic acid is present at about 1.0 μM to about 1000 mM, and lipids are present at about 0.1 mL of lipid concentrate/L to about 10 mL of lipid concentrate/L.

6. The medium of claim 5, wherein sodium bicarbonate is present at about 1.725 g/L, hepes is present at about 4.9 mM, serum albumin is present at about 4.0 g/L, fetuin is present at about 1.0 g/L, α₂-macroglubulin is present at about 4 mg/L, hydrocortisone is present at about 100 nM, ascorbic acid is present at about 198 μM, and lipids are present at about 1.0 mL of lipid concentrate/L.

7. The medium of claim 1, wherein bFGF is present at about 0.01 μg/L to about 100 μg/L, and TGF-β1 is present at about 0.01 μg/L to about 100 μg/L.

8. The medium of claim 7, wherein bFGF is present at about 0.1 μg/L to about 20 μg/L, and TGF-β1 is present at about 0.1 μg/L to about 20 μg/L.

9. The medium of claim 8, wherein bFGF is present at about 2.0 μg/L, and TGF-β1 is present at about 1.0 μg/L.

10. The medium of claim 1, wherein transferrin is present at about 0.01 mg/L to about 100 mg/L, insulin is present at about 0.01 mg/L to about 100 mg/L, putrescine is present at about 0.01 mg/L to about 100 mg/L, and progesterone is present at about 0.001 μg/L to about 100 μg/L.

11. The medium of claim 10, wherein transferrin is present at about 10 mg/L to about 40 mg/L, insulin is present at about 10 mg/L to about 40 mg/L, putrescine is present at about 5 mg/L to about 20 mg/L, and progesterone is present at about 0.1 μg/L to about 20 μg/L.

12. The medium of claim 11, wherein transferrin is present at about 25 mg/L, insulin is present at about 23 mg/L, putrescine is present at about 9.0 mg/L, and progesterone is present at about 5.66 μg/L.

13. The medium of claim 1, wherein said medium is filter sterilized.

14. A cell culture container comprising at least one cell and the medium of claim 1.

15. The cell culture container of claim 14, wherein said cell is a mesenchymal stem cell, also known as mesenchymal stromal cell, multipotent stromal cell, multipotent mesenchymal stromal cell, mesenchymal progenitor cell, or colony forming unit-fibroblast.

16. The cell culture container of claim 14, wherein said container is a dish, flask, vessel, bottle or multi-well plate.

17. The cell culture container of claim 14, wherein said container is coated with a protein.

18. A method of culturing a mesenchymal stem cell comprising the steps of:
(a) providing a mesenchymal stem cell or mesenchymal stem cell-containing population in culture medium in a container; and
(b) culturing said mesenchymal stem cell population in the culture medium of claim 1 under conditions that produce a monolayer of cells adhered to a surface

19. The method of claim 18, wherein said mesenchymal stem cell or mesenchymal stem cell-containing population retains a mesenchymal stem or progenitor cell marker.

20. The method of claim 18, wherein said mesenchymal stem cell or mesenchymal stem cell-containing population is obtained from bone marrow and other tissues such as adipose tissue.

21. The method of claim 18, wherein said mesenchymal stem cell or mesenchymal stem cell-containing population is passaged 1, 2, 3, 4, 5, 6, 7, 8, 9, 10, 12, 14, 16, 18, 20, 22 times or more.

22. The method of 18, wherein said mesenchymal stem cell or mesenchymal stem cell-containing population is maintained in culture for 2, 3, 4, 5, 6, 7, 8, 9, 10, 20, 25, 30, 35, 40, 45, 50, 60, 70, 80, 90 days or more.

23. The method of claim 18, further comprising inducing differentiation of said mesenchymal stem cell or mesenchymal stem cell-containing population.

24. The method of claim 23, wherein said mesenchymal stem cell or mesenchymal stem cell-containing population differentiates into cells of the adipogenic lineage and/or osteogenic lineage and/or chondrogenic lineage.

25. The method of claim 23, wherein said mesenchymal stem cell or mesenchymal stem cell-containing population differentiates into an adipocyte(s) and/or an osteoblast(s) and/or a chondroblast(s).

26. The method of claim 18, wherein said mesenchymal stem cell or mesenchymal stem cell-containing population is maintained at about 75-99% viability.

27. The method of claim 18, wherein said mesenchymal stem cell or mesenchymal stem cell-containing population is maintained at about 90-99% viability.

28. The method of claim 18, wherein said mesenchymal stem cell or mesenchymal stem cell-containing population is cultured in a stationary phase.

29. The method of claim 18, wherein cell-fold expansion is 1-10²⁰ for the first 60 days or more.
